(19) United States
(12) Patent Application Publication
(10) Pub. No.: US 2014/0363477 A1
(43) Pub. Date: Dec. 11, 2014

(54) Vegan Emulsion

(71) Applicant: Planaturo GmbH & Co. KG, Hamburg (DE)

(72) Inventor: Jürgen Starck, Glinde (DE)

(73) Assignee: PLANATURO GmbH & CO. KG, Hamburg (DE)

(21) Appl. No.: 14/358,272

(22) PCT Filed: Nov. 15, 2012
(86) PCT No.: PCT/EP2012/004740

§ 371 Date: Aug. 8, 2014

(30) Foreign Application Priority Data
Nov. 15, 2011 (DE) 102011118500.7

(51) Int. Cl. A23D 7/005; A23D 7/04; A61Q 19/00; A61K 8/92; A61K 8/06

(52) U.S. Cl. CPC A23D 7/0053; A61K 8/922; A61K 8/066; A61Q 19/00; A23D 7/04; A61K 2800/20
USPC 424/401; 514/783; 514/1.1; 424/94.1; 426/602; 426/238; 426/250

(57) ABSTRACT

The present invention relates to a vegan emulsion, in particular a food emulsion and/or a cosmetics emulsion. The emulsion according to the invention is a multiple emulsion in which at least 90% of the emulsion droplets of the multiple emulsion have a diameter of 2-10 μm, preferably 4-8 μm.

The present invention relates to a vegan emulsion, in particular a food emulsion and/or a cosmetic emulsion, as well as a production method for this emulsion. The emulsion according to the invention is a multiple emulsion in which at least 90% of the emulsion droplets of the multiple emulsion have a diameter of 2-10 μm, and preferably 4-8 μm.

### BACKGROUND OF THE INVENTION

Comparable emulsions from the prior art are known, in particular from the fields of foods or cosmetics. Known food emulsions are for example milk, mayonnaises, salad dressings, or margarines or ice cream as well. In the field of cosmetics, creams or lotions are known in particular.

Many of the known emulsions cannot be designated vegan, however, since these frequently have components such as milk or whey proteins or egg yolk, or emulsifiers derived from egg yolk such as egg lecithin. Such emulsions are highly unsuitable for a vegan lifestyle—a complete renunciation of animal products. Frequently, such emulsions therefore contain soy lecithin as the emulsifier which can, however, be disadvantageous due to the origin—derived from soybeans—since soy lecithin frequently has other soy proteins that can act as allergens and trigger allergies.

Vegan emulsions such as freshly mixed salad dressings consisting of an aqueous phase and oil are frequently not particularly stable; that is, emulsion and/or phase separation occurs. In other words, the earlier finely distributed phases of the emulsion separate into an aqueous phase and oil phase.

### BRIEF SUMMARY OF THE INVENTION

The object of the present invention is therefore to create a vegan emulsion that is especially stable over the long term and in storage, and in particular does not have to be cooled to be sold in retail. The long-term stability of an emulsion which in this case means a storage time of approximately a few months, and preferably at least two to twelve months, is particularly relevant especially in the case of uncooled food emulsions, as well as emulsions in cosmetics. Another object of the invention can be to provide a vegan, low-cholesterol and low-allergy food emulsion.

Another object of the invention is to provide a production method for a vegan emulsion that is stable over the long term and in storage. Another object can be to produce a particularly homogenous emulsion that substantially retains its stability and/or structure, especially even after a pasteurization step. An object according to the invention can hence also be to improve the microbiological quality of a vegan emulsion.

### BRIEF DESCRIPTION OF THE DRAWINGS

FIG. 1 shows an emulsion droplet of a multiple emulsion.

FIG. 2 shows a cross-section of a perforated foil on which an emulsion droplet of the second emulsion of the multiple emulsion forms.

### DETAILED DESCRIPTION OF THE INVENTION

The objects according to the invention are achieved with a vegan emulsion having the features of claim 1, or a production method with the features of claim 9. Preferred embodiments of the invention are specified in the dependent claims.

According to the invention, a vegan emulsion consisting of at least one vegetable oil, water and at least one vegetable auxiliary agent is characterized in that the emulsion is a multiple emulsion, wherein at least 90% (numeric distribution) of the emulsion droplets of the multiple emulsion have a diameter of 2-10 μm, and preferably 4-8 μm.

The designation vegan generally identifies products that do not have any components originating from animals. This means in particular that no product components have an animal origin. In particular, vegan emulsions therefore do not contain any components that are obtained from animals or animal products such as milk, whey or eggs. An emulsion designates a finely distributed mixture of two normally non-miscible liquids, especially water and oil. Known emulsions are for example milk, mayonnaises, salad dressings, or margarines or ice cream as well. The vegan emulsion according to the invention can be used as a starting material for a vegan food emulsion such as a salad dressing, a “mayonnaise-like” product, a sauce, a vegan ice cream or bread spread or similar products. In a different embodiment, the vegan emulsion can be used as a starting material of a cosmetic such as a cream, lotion or shower gel or similar products.

The emulsion according to the invention consists of at least one vegetable oil such as sunflower seed, olive, canola, linseed or palm oil. Other vegetable oils can also be used, however. It is preferable to use low-allergen and/or low-cholesterol vegetable oils if possible. Low-allergen in this context means that basically no substances, especially proteins, triggering allergies, in particular widespread food allergies, are included. Widespread food allergies are in particular nut allergies (peanut, walnut, sesame seed and almonds), egg yolk and egg protein allergies, or milk allergies such as lactose intolerance, as well as allergies to types of grains or soybeans. Vegetable oils and vegetable auxiliary agents are preferably used to produce an emulsion according to the invention that does not trigger any of the aforementioned food allergies. In one particularly preferred embodiment, the emulsion therefore does not have any soy proteins and/or known emulsifiers derived from soy, especially no soy lecithin. Vegan emulsions, in particular mayonnaise-like products, frequently have a taste disadvantage in comparison to emulsions such as mayonnaise with egg yolk as the emulsifier since known vegan emulsions generally use soy-based emulsifiers that frequently also have a bitter substance originating from the soy which accordingly enters the final product. In particular by avoiding soy as an additive, a taste advantage can be achieved in comparison to previous vegan emulsions based on soy.

The at least one vegetable auxiliary agent according to the invention can in particular be selected from the group of vegetable emulsifiers, vegetable thickening agents or vegetable flavors such as from the group of spices. Powdered mustard can preferably be used as the vegetable emulsifier. This powdered mustard can preferably be ground very fine, that is, the powdered mustard particles are preferably not greater than 2 to 20 μm, especially preferably not greater than 2 to 10 μm or 4 to 8 μm in a sieve analysis. Vegetable thickening agents can be locust bean gum flour, carrageenan, guar gum flour, alginates, microcrystalline cellulose, methylcellulose or other cellulose derivatives. Alternately, thickening agents such as xanthan gum, gellan gum or agar, or other thickening agents produced from bacteria or obtained from bacteria can be used. The particle size of the vegetable auxiliary agents can for example be determined with sieves with a corresponding mesh width.

The emulsion according to the invention is a multiple emulsion. A multiple emulsion can for example be a water/oil/water emulsion, or an oil/water/oil emulsion. All multiple emulsions consist of at least one inner first emulsion and one outer second emulsion. The emulsion according to the invention is preferably a water/oil/water emulsion. By using a water/oil/water emulsion, various advantages can be achieved. For example, the amount of vegetable oil in the emulsion can accordingly be reduced. Reducing the amount of oil can mean more than that the emulsion has less fat or oil and that the emulsion is accordingly lower in calories; in addition, oil which is expensive in comparison to water can be saved, and a reduction in the cost of materials can accordingly be achieved.

Furthermore, particularly advantageous applications and effects can be realized by using a multiple emulsion. With a water/oil/water emulsion, it can for example be advantageous to dissolve flavors, dyes or biologically active substances in the inner, aqueous phase of the first emulsion. Such an inner phase is generally protected very well in multiple emulsions since the aqueous phase is within an emulsion droplet of the multiple emulsion. Active substances or active ingredients that are thus e.g. protected from oxidation can hence preferably be in a water/oil/water emulsion, such as in the cosmetics field.

According to the invention, at least 90%—meaning a numeric distribution—of the emulsion droplets of the multiple emulsion can have a diameter of 2-10 μm and preferably 4-8 μm. The emulsion droplet diameter distribution preferably basically corresponds to a Gaussian normal distribution. That is, most emulsion droplets have a diameter of 5-7 μm. In considering such a normal distribution, a peak of the bell curve would for example be found at a diameter of approximately 5-7 μm.

Particularly preferably, at least 90% of the emulsion droplets have a diameter of 3-7 μm so that most of the emulsion droplets are approximately 5 μm large.

Preferably, the vegan emulsion, in particular the second emulsion of the multiple emulsion, can be produced by using a metal perforated foil, or by a textile structure. It would also be conceivable, however, to produce the at least second emulsion of the multiple emulsion by means of a ceramic membrane such as a crossflow membrane. The second emulsion is preferably produced by means of a perforated foil having holes with a diameter of 2-10 μm, or a fabric having a mesh width of 2 to 10 μm. Preferably, the perforated foil, fabric or ceramic membrane is designed and/or adapted to the production method so that a substantially continuous production process and/or sterilization or cleaning is possible. The perforated foil and/or the fabric preferably consists of a metal material that can be steam-sterilized. Particularly preferably, the perforated foil, fabric or ceramic membrane is designed so that the liquids which flow through, in particular the first emulsion, adhere as little as possible or generate as little friction as possible, for example because they have a nanostructure, in particular similar to a lotus leaf.

A method according to the invention also exists for producing a vegan emulsion, in particular according to claim 1, wherein the method has at least the following steps: a) Production of a first emulsion from oil and water, b) Dispersiton of the vegetable auxiliary agents in an aqueous phase or in the first emulsion, c) Production of a multiple emulsion from the first emulsion and the aqueous phase, characterized in that at least 90% of the emulsion droplets of the multiple emulsion have a diameter of 2-10 μm, and preferably 4-8 μm.

In a first step of the production method according to the invention, a first emulsion is produced consisting of in particular a vegetable oil and water. The first emulsion can in particular be produced by means of ultrasound, or by means of a homogenizer. “By means of ultrasound” can mean that an ultrasonic emitter emits sound waves in the aqueous phase and/or the oil, and the sound waves cause a substantially even, fine distribution of the liquids giving rise to an emulsion. A homogenizer can in particular be a mixing device such as an Ultrathurax homogenizer that mixes the two phases by means of a mechanical mixing process to generate an emulsion.

In another step, at least one vegetable auxiliary agent is dispersed in the aqueous phase or in the first emulsion. Vegetable auxiliary agents are in particular those that are used for the vegan emulsion according to the invention. Preferably already during step a), the production step, a flavor carrier, an aroma, a dye or a biologically active substance is added to the first emulsion. Biologically active substances in this context can be active ingredients, in particular from cosmetics, and/or substances that are supposed to have a cosmetic effect for example on the skin. Vitamins, proteins such as enzymes, cofactors, amino acids or cholesterol-lowering active ingredients are also conceivable. In a next step c), the first emulsion passes through, in particular, a metal perforated foil, a fabric structure such as a sieve, or a ceramic membrane. The multiple emulsion is then formed in particular while exiting into the aqueous phase. Another step d) can follow the method step c). In this step, the multiple emulsion can be heat treated, such as pasteurized.

Additional steps can be added to the production method. In particular, the emulsion according to the invention, preferably a food emulsion, can be mixed with other additives while being processed into an end product, in particular flavors such as salt, sugar or spices, or organic acids such as acetic acid, citric acid or ascorbic acid.

These and other features and advantages of the invention are described in greater detail with reference to the attached drawings of an exemplary embodiment of production steps according to the invention. The drawings show:

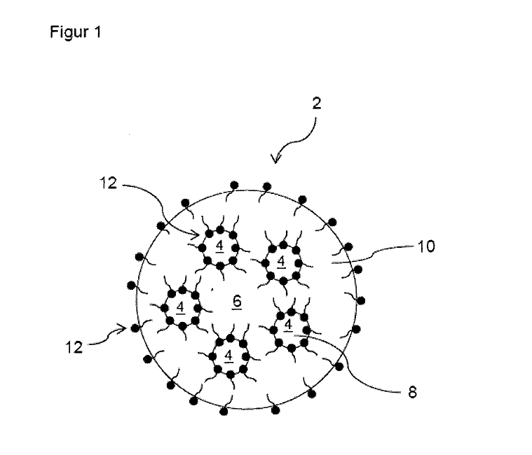

FIG. 1 shows an emulsion droplet 2 of a multiple emulsion. According to the invention, at least 90% of the emulsion droplets have a diameter of 2-10 μm and preferably 4-8 μm. The emulsion droplet 2 of the multiple emulsion has an inner emulsion droplet 8 as a first emulsion that has an inner aqueous phase 4 and an outer oil phase 6. This first oil/water emulsion is produced in step a) of the production method. In step c), the second, outer emulsion is produced. The emulsion droplets 10 of the second emulsion have an inner oil phase 6 that is simultaneously the outer oil phase 6 of the first emulsion. The outer phase of the emulsion droplet 10 is an aqueous phase (not shown) that encloses the emulsion droplet 10. The emulsion droplet 10 of the second emulsion is as shown simultaneously an emulsion droplet 2 of the multiple emulsion. Emulsifying auxiliary agents 12 are preferably disposed at the boundary surfaces between an oil phase 6 and an aqueous phase 4.

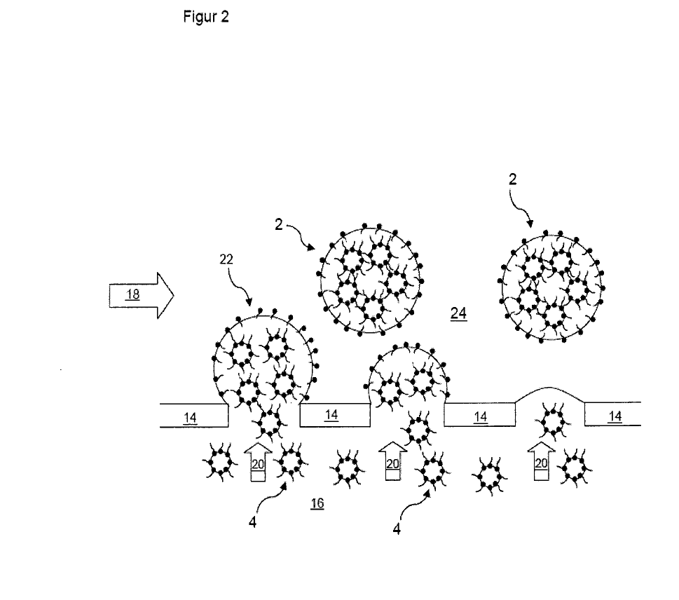

FIG. 2 shows a cross-section of a perforated foil 14 on which an emulsion droplet 2 of the second emulsion of the multiple emulsion 24 forms. The emulsion droplets 2 are formed during and/or after passing through the perforated foil 14. In an alternative embodiment, a fabric or a ceramic membrane can be used instead of the perforated foil. The structure characterized by reference sign 14 can therefore also be a fabric or a membrane.

The first emulsion 16, an oil/water emulsion, flows as shown by the block arrows 20 out of a recipient vessel through the perforated foil, fabric or membrane 14, and is then mixed with an aqueous phase. As indicated by the block arrow 18, the aqueous phase flows through the perforated foil, fabric or membrane 14. At the boundary surface between the first emulsion 16 and the aqueous phase, an emulsion droplet 2 forms as indicated with reference sign 22 so that a multiple emulsion 24, depicted as a water/oil/water emulsion here, is formed.

1. A vegan emulsion comprising at least one vegetable oil, water and at least one vegetable auxiliary agent, wherein the emulsion is a multiple emulsion, wherein at least 90% of the emulsion droplets of the multiple emulsion have a diameter of 2-10 μm.

2. The emulsion according to claim 1, wherein at least 90% of the emulsion droplets have a diameter of 4-8 μm.

3. The emulsion according to claim 1 or 2, wherein the multiple emulsion is a water/oil/water emulsion.

4. The emulsion according to claim 1 or 2, wherein the emulsion is a food emulsion and/or a cosmetic emulsion.

5. The emulsion according to claim 1 or 2, wherein the emulsion does not have any egg yolk, or any emulsifier derived from egg yolk.

6. The emulsion according to claim 1 or 2, wherein the emulsion does not have any soy protein, or any emulsifier derived from soy.

7. The emulsion according to claim 1 or 2, wherein the emulsion droplet diameter distribution substantially corresponds to a Gaussian normal distribution.

8. The emulsion according to claim 1 or 2, wherein the auxiliary agents have a particle size of 4-10 μm.

9. The emulsion according to claim 1 or 2, wherein at least the second emulsion of the multiple emulsion is produced by means of a perforated foil or by means of a fabric structure.

10. A method to produce a vegan emulsion according to claim 1, wherein the method comprises at least the following steps:
a) Production of a first emulsion from oil and water,
b) Dispersion of at least one vegetable auxiliary agent in an aqueous phase, or in the first emulsion,
c) Production of a multiple emulsion from the first emulsion and the aqueous phase, wherein at least 90% (numeric distribution) of the emulsion droplets of the multiple emulsion have a diameter of 2-10 μm.

11. The method according to claim 10, wherein at least the first emulsion is produced by means of ultrasound or means of a homogenizer.

12. The method according to claim 10 or 11, wherein a flavor carrier and/or a dye and/or a biologically active substance is added to the production step a) for the first emulsion.

13. The method according to claim 10 or 11, in which further in step c), the first emulsion passes through a perforated foil, a fabric structure or a ceramic membrane, and the multiple emulsion is formed while exiting into the aqueous phase.

14. The method according to claim 10 or 11 which comprises the further step:
d) Pasteurization of the multiple emulsion.

15. The emulsion according to claim 8, wherein the auxiliary agents have a particle size of 4-8 μm.

16. The method according to claim 10, wherein the emulsion droplets of the multiple emulsion have a diameter of 4-8 μm.

17. The method according to claim 13 which comprises the further step:
d) Pasteurization of the multiple emulsion.
